(19) United States
(12) Patent Application Publication
(10) Pub. No.: US 2022/0126253 A1
(43) Pub. Date: Apr. 28, 2022

(54) ACRYLAMIDE PRODUCTION SYSTEM

(71) Applicant: Guangdong Baomo Biochemical Co., Ltd., Zhuhai City (CN)

(72) Inventors: Wang WEI, Zhuhai City (CN); Siquan ZHANG, Zhuhai City (CN); Dechao WU, Zhuhai City (CN); Yuanqiang FAN, Zhuhai City (CN); Bo MI, Zhuhai City (CN)

(73) Assignee: Guangdong Baomo Biochemical Co., Ltd., Zhuhai City (CN)

(21) Appl. No.: 17/332,083

(22) Filed: May 27, 2021

(30) Foreign Application Priority Data
Oct. 27, 2020 (CN) 202011160821.3

(51) Int. Cl. B01J 19/00; C07C 231/24

(52) U.S. Cl. CPC B01J 19/0066; B01F 7/18; C07C 231/24; B01J 19/0013

(57) ABSTRACT

The present disclosure provides an acrylamide production system. The acrylamide production system includes a storage device, a reaction kettle, a filter device, an impurity removal device, and a collection device. The storage device is used for storing microbes required for a reaction. A discharge end of the storage device is connected to a feed end of the reaction kettle through a pipeline. A discharge end of the reaction kettle is connected to a feed end of the filter device through a pipeline. A filtrate discharge end of the filter device is connected to a feed end of the impurity removal device through a pipeline. A discharge end of the impurity removal device is connected to the collection device through a pipeline. According to the acrylamide production system of the present disclosure, the whole process for producing an acrylamide aqueous solution is simple, which facilitates producing a high-purity acrylamide solution.

### PRIORITY CLAIM AND CROSS-REFERENCE

This application claims the benefit and the priority of Chinese Application No. 202011160821.3, filed Oct. 27, 2020, which application is expressly incorporated by reference herein in its entirety.

### TECHNICAL FIELD

The present disclosure relates to the technical field of acrylamide production equipment, and in particular, to an acrylamide production system.

### BACKGROUND

The traditional method for producing acrylamide in industry is to perform hydrolysis reaction on acrylonitrile by taking copper in a reduced state as a catalyst. At present, a method for producing acrylamide by using a microbial catalyst has been developed, and the method has been applied in practice. However, the method for producing acrylamide by using the microbial catalyst is not mature enough in industrial application, and the whole production process is not simple enough, so that too much manpower and time are cost, and the purity of the produced acrylamide aqueous solution is not high enough, which leads to low purity of acrylamide crystals obtained by subsequent crystallization.

### SUMMARY

The present disclosure aims to solve one of the technical problems in the prior art. Therefore, the present disclosure provides an acrylamide production system, which makes the whole production process of acrylamide simple, and facilitates producing high-purity acrylamide aqueous solution.

The acrylamide production system according to the embodiment of the present disclosure includes a storage device, a reaction kettle, a filter device, an impurity removal device, and a collection device. The storage device is used for storing microbes required for a reaction. A discharge end of the storage device is connected to a feed end of the reaction kettle through a pipeline. A discharge end of the reaction kettle is connected to a feed end of the filter device through a pipeline. A filtrate discharge end of the filter device is connected to a feed end of the impurity removal device through a pipeline. A discharge end of the impurity removal device is connected to the collection device through a pipeline.

The acrylamide production system according to the embodiment of the present disclosure at least has the following beneficial effects: nitrile hydratase-containing microbes stored in the storage device are transferred to the reaction kettle through a transfer pump, and then acrylonitrile and other required materials are added into the reaction kettle to perform a hydrolysis reaction in the reaction kettle, so the acrylonitrile is converted into acrylamide under the action of the microbial catalyst. After the reaction is ended, a reaction liquid enters the filter device to remove the catalyst and other impurities remaining in the reaction liquid, and then the filtered reaction liquid is introduced into the impurity removal device to remove impurity ions from the reaction liquid. Finally, the high-purity acrylamide aqueous solution is obtained, and the acrylamide solution is stored in the collection device. The whole production process is simple, much manpower and time can be saved, and the purity of the produced acrylamide aqueous solution is high.

According to some embodiments of the present disclosure, the outside wall of the storage device is wound with a coil pipe. A liquid inlet is formed in one end of the coil pipe, and a liquid outlet is formed in the other end of the coil pipe.

According to some embodiments of the present disclosure, a temperature measuring element is arranged on the outside wall of the storage device. A temperature measuring point of the temperature measuring element is arranged inside the storage device.

According to some embodiments of the present disclosure, the reaction kettle includes: a stirring rod. One end of the stirring rod is arranged outside the reaction kettle and is provided with a driving part, and the other end of the stirring rod extends into the reaction kettle and is provided with a stirring part in the extension direction.

According to some embodiments of the present disclosure, the stirring part includes a plurality of groups of stirring components. Each group of stirring components includes at least two stirring paddles. One end of each stirring paddle is connected to the stirring rod. Every two adjacent groups of stirring components are arranged at an interval in the extension direction of the stirring rod.

According to some embodiments of the present disclosure, the filter device includes: a water inlet pipe, a water outlet pipe, a plurality of filter pipes, a first branch pipe, and a second branch pipe. A water inlet end of the water inlet pipe is connected to the discharge end of the reaction kettle through a pipeline. A water outlet end of the water outlet pipe is connected to the feed end of the impurity removal device. The interior of each filter pipe is filled with a filter component. A first input end of the first branch pipe is connected to a first output end of the water inlet pipe. The feed end of each filter pipe is connected to an output end of the first branch pipe through a pipeline. A filtrate discharge end of each filter pipe is connected to an input end of the second branch pipe through a pipeline. A first output end of the second branch pipe is connected to a first input end of the water outlet pipe.

According to some embodiments of the present disclosure, the filter device further includes: a first solenoid valve, arranged between a first input end of the water inlet pipe and a first input end of the first branch pipe; a second solenoid valve, arranged between a second input end of the first branch pipe and a second input end of the water outlet pipe; a third solenoid valve, arranged between a second output end of the water inlet pipe and a second output end of the second branch pipe; a fourth solenoid valve, arranged between a first output end of the second branch pipe and a first input end of the water outlet pipe.

According to some embodiments of the present disclosure, the filter components are hollow fiber ultrafiltration membranes.

According to some embodiments of the present disclosure, the filter device further includes: a waste liquid outlet. A waste liquid discharge end of each filter pipe is connected to the waste liquid outlet through a pipeline.

According to some embodiments of the present disclosure, the impurity removal device includes: a cation bed and an anion bed. A feed end of the cation bed is connected to the filtrate discharge end of the filter device. A discharge end of the cation bed is connected to a feed end of the anion bed. A discharge end of the anion bed is connected to the collection device.

Additional aspects and advantages of the present disclosure will be set forth in part in the following description. Part will become apparent from the following description, or will be understood by the practice of the present disclosure.

### BRIEF DESCRIPTION OF THE DRAWINGS

The above and/or additional aspects and advantages of the present disclosure will become apparent and comprehensible from the description of the embodiments in combination with the accompanying drawings, where

FIG. 1 is a structural schematic diagram of an acrylamide production system of the embodiment of the present disclosure;

FIG. 2 is a structural schematic diagram of a storage device of the embodiment of the present disclosure;

FIG. 3 is a structural schematic diagram of a reaction kettle of the embodiment of the present disclosure;

FIG. 4 is a structural schematic diagram of a stirring part of the reaction kettle shown in FIG. 3;

FIG. 5 is a structural schematic diagram of a filter device of the embodiment of the present disclosure.

### REFERENCE SIGNS IN THE DRAWINGS

100—storage device; 110—coil pipe; 120—liquid inlet; 130—liquid outlet; 140—temperature measuring element; 200—reaction kettle; 210—stirring rod; 220—driving part; 230—stirring paddle; 240—feeding port; 241—feeding port cover; 250—transparent window; 300—filter device; 310—water inlet pipe; 320—water outlet pipe; 330—filter pipe; 340—first branch pipe; 350—second branch pipe; 360—first solenoid valve; 370—second solenoid valve; 380—third solenoid valve; 390—fourth solenoid valve; 391—waste liquid outlet; 400—impurity removal device; 410—cation bed; 420—anion bed; 500—collection device.

### DETAILED DESCRIPTION

This part will describe specific embodiments of the present disclosure in detail. Better embodiments of the present disclosure is shown in the accompanying drawings. The function of the accompanying drawings is to supplement the description of the text part of the specification with graphics, so that people can intuitively and vividly understand each technical feature and the whole technical solution of the present disclosure, but it cannot be construed as a limitation to the scope of protection of the present disclosure.

In the descriptions of the present disclosure, it should be understood that orientations or positional relationships involving the description of orientations, for example, “upper”, “lower”, “front”, “rear”, “left”, “right”, etc. are the orientations or positional relationships shown based on the accompanying drawings, which are merely used for facilitating describing the present disclosure and simplifying the description, rather than indicating or implying that devices or elements must have particular orientations, and constructed and operated in particular orientations. Thus, it cannot be construed as a limitation to the present disclosure.

In the description of the present disclosure, “a plurality” means one or more; multiple means more than two; greater than, less than, more than, etc. are construed as excluding the number; above, below, within, etc. are construed as including the number. The descriptions of terms “first”, “second”, “third”, and “fourth” are merely used for distinguishing technical features, but cannot be construed as indicating or implying relative importance or implicitly indicating the number of indicated technical features or implicitly indicating the precedence relationship of the indicated technical features.

As used herein, the term “connected to” is understood to encompass terms “fluidly coupled to” or “fluidly coupled with,” and will be understood that the components or units are configured to have fluid, liquid, or gas flow through the components.

In the description of the present disclosure, unless otherwise specified, the words, such as arranged, installed and connected, shall be interpreted broadly. Those skilled in the art can reasonably determine specific meanings of the above words in the present disclosure in combination with the specific content of the technical solution.

As shown in FIG. 1, the acrylamide production system according to the embodiment of the present disclosure includes a storage device 100, a reaction kettle 200, a filter device 300, an impurity removal device 400, and a collection device 500. The storage device 100 is used for storing microbes required for a reaction. A discharge end of the storage device 100 is connected to a feed end of the reaction kettle 200 through a pipeline. A discharge end of the reaction kettle 200 is connected to a feed end of the filter device 300 through a pipeline. A filtrate discharge end of the filter device 300 is connected to a feed end of the impurity removal device 400 through a pipeline. A discharge end of the impurity removal device 400 is connected to the collection device 500 through a pipeline.

Specifically, the temperature inside the storage device 100 is set to be the temperature suitable for the preservation of the microbes. The selected microbes are the microbes, such as Nocardia, Corynebacterium, Bacillus or Micrococcus, that produce or retain nitrile hydratase. The microbes serve as a catalyst for hydrolysis reaction of acrylonitrile. Before reaction, the microbes are transferred into the reaction kettle 200 through a transfer pump first, and then acrylonitrile and other required materials are added into the reaction kettle 200 to perform the hydrolysis reaction. The acrylonitrile is converted into acrylamide under the action of the microbial catalyst. After the reaction is ended, a reaction liquid is pressed into the filter device 300 through the transfer pump, and remaining catalyst and other byproducts are removed to obtain a coarse acrylamide solution through the filter device 300. The coarse acrylamide solution is transferred into the impurity removal device 400 through the transfer pump to remove cation and anion impurities from the solution to obtain a high-purity acrylamide solution, and the acrylamide solution is stored in the collection device 500. The whole production process is simple and quick, much manpower and time can be saved, and high-purity acrylamide solution can be produced.

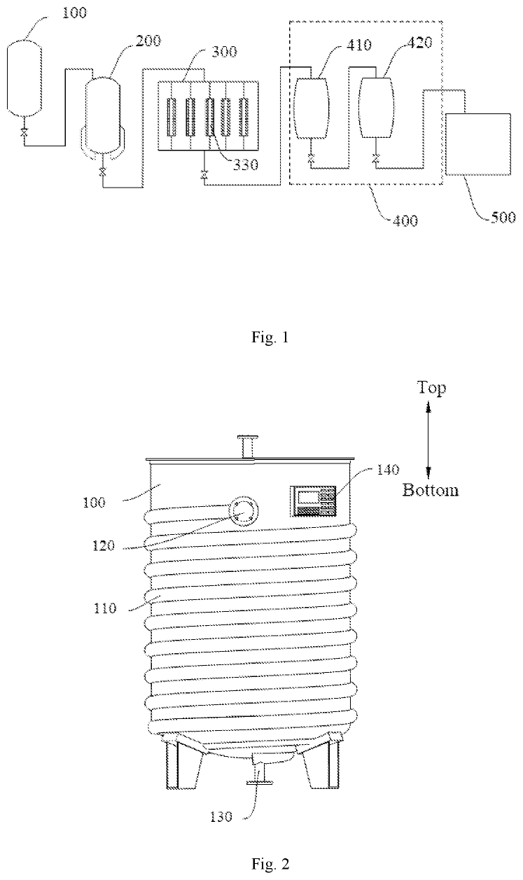

As shown in FIG. 2, in some embodiments of the present disclosure, the outside wall of the storage device 100 is wound with a coil pipe 100. A liquid inlet 120 is formed in one end of the coil pipe 110, and a liquid outlet 130 is formed in the other end of the coil pipe 110. The liquid inlet 120 and the liquid outlet 130 can be connected to a water tank through a circulating pump, so that a liquid at a certain temperature can be introduced into the coil pipe 110. The temperature of the liquid in the water tank is set to be the temperature that is suitable for the preservation of the microbes, so that the temperature suitable for the preservation of the microbes is kept inside the storage device 100 through the coil pipe 110.

As shown in FIG. 2, in some embodiments of the present disclosure, a temperature measuring element 140 is arranged on the outside wall of the storage device 100. A temperature measuring point of the temperature measuring element 140 arranged in storage device 100. The temperature measuring element 140 may adopt a temperature sensor. The temperature inside the storage device 100 is monitored in real time by using the temperature measuring element 140, which facilitates adjusting the temperature inside the storage device 100 by changing the temperature of the liquid circulating in the coil pipe 110.

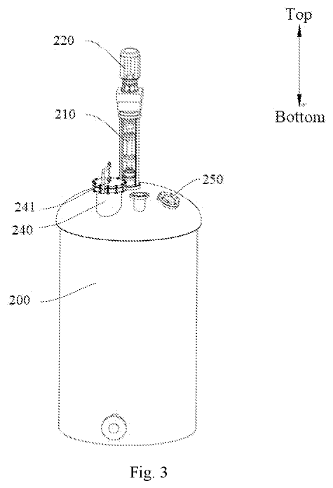

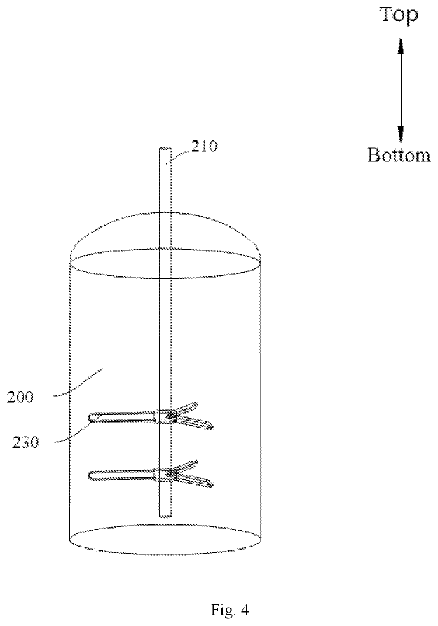

As shown in FIG. 3 and FIG. 4, in some embodiments of the present disclosure, the reaction kettle 200 includes: a stirring rod 210. One end of the stirring rod 210 is arranged outside the reaction kettle 200 and is provided with a driving part 220, and the other end of the stirring rod 210 extends into the reaction kettle 200 and is provided with a stirring part in the extension direction. Specifically, the driving part 220 may adopt a motor. An output shaft of the motor is connected to the stirring rod 210. The stirring rod 210 can be driven to rotate through the motor. The stirring rod 210 drives the stirring part to stir, so that the materials in the reaction kettle 200 are mixed more fully, thereby accelerating the reaction speed.

As shown in FIG. 4, in a further embodiment of the present disclosure, the stirring part includes a plurality of groups of stirring components. Each group of stirring components includes at least two stirring paddles 230. One end of each stirring paddle 230 is connected to the stirring rod 210. Every two adjacent stirring components 210 are arranged at an interval in the extension direction of the stirring rod 210. The materials at different heights can be stirred simultaneously by arranging the plurality of groups of stirring components, so that the materials are stirred more fully, thereby accelerating the reaction speed.

As shown in FIG. 3, in some embodiments of the present disclosure, a feeding port 240 is formed in the upper end of the reaction kettle 200. A detachable feeding port cover 241 is further arranged at the upper end of the feeding port 240, which facilitates adding the materials required for the reaction into the reaction kettle 200 from the feeding port 240. A transparent window 250 is further arranged in the upper end of the reaction kettle 200, which facilitates observing the reaction situations inside the reaction kettle 200.

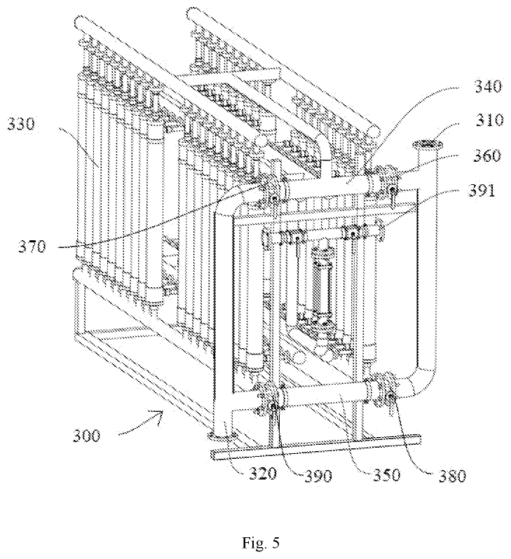

As shown in FIG. 5, in some specific embodiments of the present disclosure, the filter device 300 includes: a water inlet pipe 310, a water outlet pipe 320, a plurality of filter pipes 330, a first branch pipe 340, and a second branch pipe 350. A water inlet end of the water inlet pipe 310 is connected to a discharge end of the reaction kettle 200 through a pipeline. A water outlet end of the water outlet pipe 320 is connected to the feed end of the impurity removal device 400 through a pipeline. The interior of each filter pipe 330 is filled with a filter component. The feed end of each filter pipe 330 is connected to an output end of the first branch pipe 340 through a pipeline. A first input end of the first branch pipe 340 is connected to a first output end of the water inlet pipe 310. A filtrate discharge end of each filter pipe 330 is connected to an input end of the second branch pipe 350 through a pipeline. A first output end of the second branch pipe 350 is connected to a first input end of the water outlet pipe 320.

Specifically, the reaction liquid obtained after the reaction in the reaction kettle 200 is ended is transferred into the filter device 300 through the transfer pump. The reaction liquid enters the filter pipes 330 from the feed ends of the filter pipes 330 after passing through the water inlet pipe 310 and a first branch pipe 340. The filter components in the filter pipes 330 filter away microbes and some byproducts remaining in the reaction liquid, so that the reaction liquid is divided into a filtrate and a waste liquid. The filtrate is a coarse acrylamide solution. The coarse acrylamide solution comes out from the filtrate discharge ends of the filter pipes 330, and is transferred into the impurity removal device 400 through the second branch pipe 350 and the water outlet pipe 320, so as to perform further impurity removal.

As shown in FIG. 5, in a further embodiment of the present disclosure, the filter device 300 further includes a first solenoid valve 360, a second solenoid valve 370, a third solenoid valve 380, and a fourth solenoid valve 390. A first input end of the first branch pipe 340 is connected to a first output end of the water inlet pipe 310 through the first solenoid valve 360. A second input end of the first branch pipe 340 is connected to a second input end of the water outlet pipe 320 through the second solenoid valve 370. A second output end of the water inlet pipe 310 is connected to a second output end of the second branch pipe 350 through the third solenoid valve 380. A first output end of the second branch pipe 340 is connected to a first input end of the water outlet pipe 320 through the fourth solenoid valve 390.

Specifically, when only the first solenoid valve 360 and the fourth solenoid valve 390 are opened, the reaction liquid enters the first branch pipe 340 from the water inlet pipe 310 through the first solenoid valve 360, and then is filtered through the filter pipes 330. The filtrate comes out from the filtrate discharge ends of the filter pipes 330, enters the second branch pipe 350, then enters the water outlet pipe 320 through the fourth solenoid valve 390, and is transferred into the impurity removal device 400. After the filter pipes 330 are used for a period of time, impurities, such as dirt, will accumulate on the filter components to affect the filtering effect, so it is necessary to perform back flush on the filter pipes 330. When the filter pipes 330 are subjected to back flush, only the second solenoid valve 370 and the third solenoid valve 380 are opened, clean water enters from the water inlet pipe 310, then enters the second branch pipe 350 through the third solenoid valve 380, then enters the filter pipes 330 through the filtrate discharge ends of the filter pipes 330 to perform back flush on the filter pipes 330, comes out from the feed ends of the filter pipes 330, enters the first branch pipe 340, and flows out from the water outlet pipe 320 through the second solenoid valve 370. The back flush operation of the filter device 300 is realized through the above operation.

In some specific embodiments of the present disclosure, the filter components are hollow fiber ultrafiltration membranes. Micropores are distributed in the walls of the hollow fiber ultrafiltration membranes. The molecular weight of the materials can be intercepted by the pore diameters is relatively large. After the reaction liquid passes through the hollow fiber ultrafiltration membranes in the filter pipes 330, the particles and macromolecules larger than the membrane pores are intercepted due to a screening action, while an acrylamide solution comes out from the filtrate discharge ends of the filter pipes 330 through hollow fiber ultrafiltration membranes and is transferred into the impurity removal device 400. The filtered impurity-containing waste liquid comes out from waste liquid discharge ends of the filter pipes 330.

Referring to FIG. 5, in some specific embodiments of the present disclosure, the filter device 300 further includes a waste liquid outlet 391. The waste liquid discharge end of each filter pipe 330 is connected to the waste liquid outlet 391 through a pipeline. A control valve is further arranged between the waste liquid discharge end and the waste liquid outlet 391. The waste liquid can be discharged from the waste liquid outlet 391 by opening the control valve.

As shown in FIG. 1, in some specific embodiments of the present disclosure, the impurity removal device 400 includes a cation bed 410 and an anion bed 420. A feed end of the cation bed 410 is connected to the filtrate discharge end of the filter device 300. A discharge end of the cation bed 410 is connected to the feed end of the anion bed 420. The discharge end of the anion bed 420 is connected to the collection device 500. Specifically, the cation bed 410 is filled with cation exchange resin, which is used for removing cation impurities from the solution. The anion bed 420 is filled with anion exchange resin, which is used for removing anion impurities from the solution. Impurity ions in the solution are removed after the coarse acrylamide solution passes through the cation bed 410 and the anion bed 420, so that a high-purity acrylamide solution can be obtained, and the acrylamide solution is stored in the collection device 500.

In the description of the specification, descriptions made with reference to the terms “an embodiment”, “a further embodiment”, “some specific embodiments”, “some examples”, or the like refer to that specific features, structures, or characteristics described in combination with the embodiment or the example are included in at least one embodiment or example of the present disclosure. In the present specification, the schematic representation of the above terms does not necessarily mean the same embodiment or example. Furthermore, the specific features, structures, materials, or characteristics described may be combined in a suitable manner in any one or more embodiments or examples.

Although the embodiments of the present disclosure have been shown and described, those of ordinary skill in the art can understand that a variety of changes, modifications, substitutions, and variants can be made to these embodiments without departing from the principle and purpose of the present disclosure, and the scope of the present disclosure is defined by the claims and their equivalents.

What is claimed is:

1. An acrylamide production system, comprising:
a storage device, used for storing microbes required for a reaction;
a reaction kettle, wherein a discharge end of the storage device is connected to a feed end of the reaction kettle through a pipeline;
a filter device, wherein a discharge end of the reaction kettle is connected to a feed end of the filter device through a pipeline;
an impurity removal device, wherein a filtrate discharge end of the filter device is connected to a feed end of the impurity removal device through a pipeline;
a collection device, wherein a discharge end of the impurity removal device is connected to the collection device through a pipeline.

2. The acrylamide production system according to claim 1, wherein the outside wall of the storage device is wound with a coil pipe; a liquid inlet is formed in one end of the coil pipe; a liquid outlet is formed in the other end of the coil pipe.

3. The acrylamide production system according to claim 1, wherein a temperature measuring element is arranged on the outside wall of the storage device; a temperature measuring point of the temperature measuring element is arranged inside the storage device.

4. The acrylamide production system according to claim 2, wherein a temperature measuring element is arranged on the outside wall of the storage device; a temperature measuring point of the temperature measuring element is arranged inside the storage device.

5. The acrylamide production system according to claim 1, wherein the reaction kettle comprises a stirring rod; one end of the stirring rod is arranged outside the reaction kettle and is provided with a driving part, and the other end of the stirring rod extends into the reaction kettle and is provided with a stirring part in the extension direction.

6. The acrylamide production system according to claim 5, wherein the stirring part comprises a plurality of groups of stirring components; each group of stirring components comprises at least two stirring paddles; one end of each stirring paddle is connected to the stirring rod; every two adjacent groups of stirring components are arranged at an interval in the extension direction of the stirring rod.

7. The acrylamide production system according to claim 1, wherein the filter device comprises:
a water inlet pipe, wherein a water inlet end of the water inlet pipe is connected to a discharge end of the reaction kettle through a pipeline;
a water outlet pipe, wherein a water outlet end of the water outlet pipe is connected to the feed end of the impurity removal device;
a plurality of filter pipes, wherein the interior of each filter pipe is filled with a filter component;
a first branch pipe, wherein a first input end of the first branch pipe is connected to a first output end of the water inlet pipe, and the feed end of each filter pipe is connected to an output end of the first branch pipe through a pipeline;
a second branch pipe, wherein a filtrate discharge end of each filter pipe is connected to an input end of the second branch pipe through a pipeline, and a first output end of the second branch pipe is connected to a first input end of the water outlet pipe.

8. The acrylamide production system according to claim 7, wherein the filter device further comprises:
a first solenoid valve, arranged between a first input end of the water inlet pipe and a first input end of the first branch pipe;
a second solenoid valve, arranged between a second input end of the first branch pipe and a second input end of the water outlet pipe;
a third solenoid valve, arranged between a second output end of the water inlet pipe and a second output end of the second branch pipe;
a fourth solenoid valve, arranged between a first output end of the second branch pipe and a first input end of the water outlet pipe.

9. The acrylamide production system according to claim 7, wherein the filter components are hollow fiber ultrafiltration membranes.

10. The acrylamide production system according to claim 8, wherein the filter components are hollow fiber ultrafiltration membranes.

11. The acrylamide production system according to claim 7, wherein the filter device further comprises a waste liquid outlet; a waste liquid discharge end of each filter pipe is connected to the waste liquid outlet through a pipeline.

12. The acrylamide production system according to claim 1, wherein the impurity removal device comprises:
a cation bed, wherein a feed end of the cation bed is connected to a filtrate discharge end of the filter device;
an anion bed, wherein a discharge end of the cation bed is connected to a feed end of the anion bed, and a discharge end of the anion bed is connected to the collection device.
